(19) United States
(12) Patent Application Publication
(10) Pub. No.: US 2023/0125910 A1
(43) Pub. Date: Apr. 27, 2023

(54) METHOD FOR GENERATING A DIAGNOSIS MODEL USING BIOMARKER GROUP-RELATED VALUE INFORMATION, AND METHOD AND DEVICE FOR DIAGNOSING MULTI-CANCER USING THE SAME

(71) Applicant: KKL CONSORTIUM LIMITED, Road Town (VG)

(72) Inventor: Lancelot Fitzgerald James, Hong Kong (HK)

(21) Appl. No.: 17/511,721

(22) Filed: Oct. 27, 2021

(51) Int. Cl. G16H 50/20; G16H 50/70; G16H 50/30; A61B 5/00

(52) U.S. Cl. CPC G16H 50/20; G16H 50/70; G16H 50/30; A61B 5/7267; A61B 5/7275

(57) ABSTRACT

A method for generating a diagnosis model capable of diagnosing multi-cancer by using biomarker group-related value information, and a method and a device for diagnosing multi-cancer using the same; and more particularly, to a method for (i) generating a classification model by using total data of biomarker group-related value information, (ii) grouping the total data for each of patients by using the classification model, (iii) generating a diagnosis model by instructing the classification model to perform re-training which uses each of the grouped total data, and (iv) generating the diagnosis model, and further including a method and a device for diagnosing multi-cancer using the same.

### FIELD OF THE DISCLOSURE

The present disclosure relates to a method for generating a diagnosis model capable of diagnosing multi-cancer by using biomarker group-related value information, and a method and a device for diagnosing multi-cancer using the same; and more particularly, to the method for (i) generating a classification model by using total data of biomarker group-related value information, (ii) grouping the total data for each of patients by using the classification model, (iii) generating a diagnosis model by instructing the classification model to perform re-training which uses each of the grouped total data and thus (iv) generating the diagnosis model, and the method and the device for diagnosing multi-cancer using the same.

### BACKGROUND OF THE DISCLOSURE

Tumor metastasis represents that some portion of a tumor is detached from a part of the body of patient and moves to other parts of the body via blood, which is an important cause of cancer-related death. A general way of diagnosing a tumor status is a biopsy which detaches and examines a part of tissue in an early stage of metastasis. It is, however, not easy to determine an exact part of the body from which the tissue is removed. As an alternative way, a liquid biopsy attracting attention in recent years can detect tumor cells in a biological sample such as blood, urine, etc. derived from a patient's body. According to the liquid biopsy, a cancer in the early stage can be detected and diagnosed and additionally a progression of cancer and its corresponding cure can be monitored.

A biomarker is a measurable indicator of some biological state or condition. Biomarkers are often measured and evaluated using blood, urine, or soft tissues to examine normal biological processes, pathogenic processes, or pharmacologic responses to a therapeutic intervention. The biomarkers can detect changes in the body by using proteins, nucleic acids, and metabolites contained in biological samples.

However, since there is a limit in diagnosing cancer with a biomarker, a cancer diagnosing method using a complex biomarkers with an improvement in diagnostic sensitivity and specificity is currently used in this field.

However, in case of predicting a certain cancer by using such complex biomarkers, a specific biomarker included in the complex biomarkers may not only represent an indicator for the certain cancer but also represent another indicator for another cancer. Thus, the certain cancer may be wrongly predicted by using the complex biomarkers.

As an example, it may indicate the adenocarcinoma as a result of prediction by using biomarker group-related value information derived from the complex biomarkers, although a patient's cancer is actually squamous cell carcinoma.

Also, in predicting a cancer by using the biomarker group-related value information of the complex biomarkers, there is a possibility of acquiring a different result according to a doctor in charge.

Thus, various methodologies have been suggested to classify a cancer by using the biomarker group-related value information of the complex biomarkers.

There are two methodologies. The first one is related to fitting a model by using total data and the second one is related to fitting each different model by each group.

There is a trade-off between bias and variance in using a methodology among the two methodologies mentioned above.

In case of fitting a model by using the total data, the variance may decrease while the bias increases because one model must be generated for all the patients.

In contrast, in case of fitting each different model by each group, the bias may decrease while the variance increases because the number of patients for each group decreases.

Accordingly, the applicant of the present disclosure provides a diagnosis model that minimizes the bias and the variance and accurately predict multi-cancer by using biomarker group-related value information.

### SUMMARY OF THE DISCLOSURE

It is an object of the present disclosure to solve all the aforementioned problems.

It is another object of the present disclosure to provide a diagnosis model that minimizes the bias and the variance by using biomarker group-related value information for each of patients.

It is still another object of the present disclosure to allow a type of cancer to be predicted accurately by using the biomarker group-related value information for multi-cancer diagnosis.

It is still yet another object of the present disclosure to allow a type of cancer to be predicted accurately through a statistical discriminant analysis using biomarker group-related value information for the multi-cancer diagnosis.

It is still yet another object of the present disclosure to increase a credibility of cancer diagnosis through the statistical discriminant analysis using the biomarker group-related value information for the multi-cancer diagnosis.

In accordance with one aspect of the present invention, there is provided a method for generating diagnosis model capable of diagnosing multi-cancer using biomarker group-related value information including steps of: (a) a diagnosis model generation device, in response to acquiring n pieces of training data including the biomarker group-related value information and its corresponding Ground Truth cancer information for each of patients, generating a multi-cancer classification model that classifies multi-cancer corresponding to the biomarker group-related value information by using the training data, wherein n is an integer of 1 or more; (b) the diagnosis model generation device generating a patient clustering model which classifies the patients into any of k clusters, wherein the k is an integer of 1 or more, by referring to multi-cancer score values outputted from the multi-cancer classification model, wherein the multi-cancer score values represent results of classifying multi-cancer by using the biomarker group-related value information; and (c) the diagnosis model generation device (i) re-training the multi-cancer classification model by using each of partial training data corresponding to each of the k clusters grouped by the patient clustering model, wherein the partial training data is a part of the training data, thereby generating a first patient cancer classification model to a k-th patient cancer classification model and thus (ii) generating a diagnosis model including the multi-cancer classification model, the patient clustering model and the first patient cancer classification model to the k-th patient cancer classification model.

As one example, at the step of (a), the diagnosis model generation device (i) inputs each piece of the biomarker group-related value information from the training data into an initial classification model designed to classify multi-cancer by using the biomarker group-related value information, (ii) instructs the initial classification model to output first multi-cancer score values resulting from predicting multi-cancer by using each of the biomarker group-related value information, (iii) trains the initial classification model by using a loss function acquired by referring to (iii-1) the first multi-cancer score values and (iii-2) each piece of the Ground Truth cancer information of the training data and thus (iv) generates the multi-cancer classification model.

As one example, the initial classification model is any one of a decision tree model, a tree ensemble model, a random forest model, Bayesian network model, support vector machine model, neural network model or logistic regression model.

As one example, at the step of (b), the diagnosis model generation device (i) inputs each of the biomarker group-related value information on each of the patients into the multi-cancer classification model and instructs the multi-cancer classification model to output second multi-cancer score values resulting from predicting multi-cancer by using each of the biomarker group-related value information, (ii) inputs the second multi-cancer score values into an initial clustering model and instructs the initial clustering model to group the patients by using the second multi-cancer score values and (iii) instructs the initial clustering model to perform unsupervised learning for grouping the patients into k clusters by using a clustered distribution and thus generates the patient clustering model.

As one example, the initial classification model is any one of a K-Means Clustering model, a Mean-Shift Clustering model, a Density-Based Spatial Clustering of Applications with Noise (DBSCAN) model, an Expectation-Maximization (EM) model, Clustering using Gaussian Mixture Models (GMM), or an Agglomerative Hierarchical Clustering model.

As one example, at the step of (c), the diagnosis model generation device (i) inputs second multi-cancer score values resulting from predicting multi-cancer by using each of the biomarker group-related value information through the multi-cancer classification model into the patient clustering model and instructs the patient clustering model to group the patients into a first cluster to a k-th cluster by using the second multi-cancer score values and (ii) instructs the patient clustering model to perform re-training by using each of first data corresponding to first patients in the first cluster to k-th data corresponding to k-th patients in the k-th cluster, respectively, and thereby generates the first patient cancer classification model to the k-th patient cancer classification model.

In accordance with another aspect of the present invention, there is provided a method for diagnosing multi-cancer using biomarker group-related value information including steps of: (a) on condition that the diagnosis model generation device, (i) in response to acquiring n pieces of training data including the biomarker group-related value information and its corresponding Ground Truth cancer information for each of patients, has generated a multi-cancer classification model that classifies multi-cancer corresponding to the biomarker group-related value information by using the training data, wherein n is an integer of 1 or more (ii) has generated a patient clustering model which classifies the patients into any of k clusters, wherein the k is an integer of 1 or more, by referring to multi-cancer score values outputted from the multi-cancer classification model, wherein the multi-cancer score values represent results of classifying multi-cancer by using the biomarker group-related value information and (iii) (iii-1) has re-trained the multi-cancer classification model by using each of partial training data corresponding to each of the k clusters grouped by the patient clustering model, wherein the partial training data is a part of the training data, (iii-2) has generated a first patient cancer classification model to a k-th patient cancer classification model and thus (iii-3) has generated a diagnosis model including the multi-cancer classification model, the patient clustering model and the first patient cancer classification model to the k-th patient cancer classification model, a multi-cancer diagnosis device acquiring certain biomarker group-related value information on a certain patient; and (b) the multi-cancer diagnosis device (i) inputting the certain biomarker group-related value information into the multi-cancer classification model and instructing the multi-cancer classification model to output certain multi-cancer score values resulting from predicting multi-cancer by using the certain biomarker group-related value information, (ii) inputting the certain multi-cancer score values into the patient clustering model and instructing the patient clustering model to output certain patient cluster information on which cluster the certain patient belongs to among the first cluster to the k-th cluster by using the certain multi-cancer values and (iii) inputting the certain biomarker group-related value information into the certain patient cancer classification model, corresponding to the certain patient cluster information, among the first patient cancer classification model to the k-th patient cancer classification model and instructing certain patient cancer classification model to output certain cancer information resulting from predicting multi-cancer by using the certain biomarker group-related value information.

As one example, at the step of (a), the diagnosis model generation device has performed processes of (i) inputting each piece of the biomarker group-related value information from the training data into an initial classification model designed to classify multi-cancer by using the biomarker group-related value information, (ii) instructing the initial classification model to output first multi-cancer score values resulting from predicting multi-cancer by using each of the biomarker group-related value information, (iii) training the initial classification model by using a loss function acquired by referring to (iii-1) the first multi-cancer score values and (iii-2) each piece of the Ground Truth cancer information from the training data and thus (iv) generating the multi-cancer classification model.

As one example, at the step of (a), the diagnosis model generation device has performed processes of (i) inputting each of the biomarker group-related value information into the multi-cancer classification model and instructing the multi-cancer classification model to output second multi-cancer score values that predict multi-cancer on each of the biomarker group-related value information, (ii) inputting the second multi-cancer score values into an initial clustering model and instructing the initial clustering model to group the patients by using the second multi-cancer score values and (iii) instructing the initial clustering model to perform unsupervised learning to grouping the patients into k clusters by using an clustered distribution and thereby generates the patient clustering model.

As one example, at the step of (a), the diagnosis model generation device has performed process of (i) inputting second multi-cancer score values where multi-cancer is predicted for each of the biomarker group-related value information through the multi-cancer classification model into the patient clustering model and instructing the patient clustering model to group the patients into a first cluster to a k-th cluster by using second multi-cancer score values, (ii) instructing the patient clustering model to perform re-training by using each of the first data corresponding to first patients in the first cluster to k-th data corresponding to k-th patients in the k-th cluster, respectively, and thereby generates first patient cancer classification model to the k-th patient cancer classification model.

In accordance with still another aspect of the present invention, there is provided a diagnosis model generation device capable of diagnosing multi-cancer using biomarker group-related value information including: one or more memories that stores instructions; and one or more processors configured to execute the instructions to perform processes of (I) in response to acquiring n pieces of training data including the biomarker group-related value information and its corresponding Ground Truth cancer information for each of patients, generating a multi-cancer classification model that classifies multi-cancer corresponding to the biomarker group-related value information by using the training data, wherein n is an integer of 1 or more; (II) generating a patient clustering model which classifies the patients into any of k clusters, wherein the k is an integer of 1 or more, by referring to multi-cancer score values outputted from the multi-cancer classification model, wherein the multi-cancer score values represent results of classifying multi-cancer by using the biomarker group-related value information; and (III) (i) re-training the multi-cancer classification model by using each of partial training data corresponding to each of the k clusters grouped by the patient clustering model, wherein the partial training data is a part of the training data, thereby generating a first patient cancer classification model to a k-th patient cancer classification model and thus (ii) generating a diagnosis model including the multi-cancer classification model, the patient clustering model and the first patient cancer classification model to the k-th patient cancer classification model.

As one example, at the process of (I), the processor (i) inputs each piece of the biomarker group-related value information from the training data into an initial classification model designed to classify multi-cancer by using the biomarker group-related value information, (ii) instructs the initial classification model to output first multi-cancer score values resulting from predicting multi-cancer by using each of the biomarker group-related value information, (iii) trains the initial classification model by using a loss function acquired by referring to (iii-1) the first multi-cancer score values and (iii-2) each piece of the Ground Truth cancer information of the training data and thus (iv) generates the multi-cancer classification model.

As one example, the initial classification model is any one of a decision tree model, a tree ensemble model, a random forest model, Bayesian network model, support vector machine model, neural network model or logistic regression model.

As one example, at the process of (II), the processor (i) inputs each of the biomarker group-related value information on each of the patients into the multi-cancer classification model and instructs the multi-cancer classification model to output second multi-cancer score values resulting from predicting multi-cancer by using each of the biomarker group-related value information, (ii) inputs the second multi-cancer score values into an initial clustering model and instructs the initial clustering model to group the patients by using the second multi-cancer score values and (iii) instructs the initial clustering model to perform unsupervised learning for grouping the patients into k clusters by using a clustered distribution and thus generates the patient clustering model.

As one example, the initial classification model is any one of a K-Means Clustering model, a Mean-Shift Clustering model, a Density-Based Spatial Clustering of Applications with Noise (DBSCAN) model, an Expectation-Maximization (EM) model, Clustering using Gaussian Mixture Models (GMM), or an Agglomerative Hierarchical Clustering model.

As one example, at the process of (III), the processor (i) inputs second multi-cancer score values resulting from predicting multi-cancer by using each of the biomarker group-related value information through the multi-cancer classification model into the patient clustering model and instructs the patient clustering model to group the patients into a first cluster to a k-th cluster by using the second multi-cancer score values and (ii) instructs the patient clustering model to perform re-training by using each of first data corresponding to first patients in the first cluster to k-th data corresponding to k-th patients in the k-th cluster, respectively, and thereby generates the first patient cancer classification model to the k-th patient cancer classification model.

In accordance with still yet another aspect of the present invention, there is provided a multi-cancer diagnosis device for diagnosing multi-cancer using biomarker group-related value information including: one or more memories that stores instructions; and one or more processors configured to execute the instructions to perform processes of (I) on condition that the diagnosis model generation device, (i) in response to acquiring n pieces of training data including the biomarker group-related value information and its corresponding Ground Truth cancer information for each of patients, has generated a multi-cancer classification model that classifies multi-cancer corresponding to the biomarker group-related value information by using the training data, wherein n is an integer of 1 or more (ii) has generated a patient clustering model which classifies the patients into any of k clusters, wherein the k is an integer of 1 or more, by referring to multi-cancer score values outputted from the multi-cancer classification model, wherein the multi-cancer score values represent results of classifying multi-cancer by using the biomarker group-related value information and (iii) (iii-1) has re-trained the multi-cancer classification model by using each of partial training data corresponding to each of the k clusters grouped by the patient clustering model, wherein the partial training data is a part of the training data, (iii-2) has generated a first patient cancer classification model to a k-th patient cancer classification model and thus (iii-3) has generated a diagnosis model including the multi-cancer classification model, the patient clustering model and the first patient cancer classification model to the k-th patient cancer classification model, acquiring certain biomarker group-related value information on a certain patient; and (II) (i) inputting the certain biomarker group-related value information into the multi-cancer classification model and instructing the multi-cancer classification model to output certain multi-cancer score values resulting from predicting multi-cancer by using the certain biomarker group-related value information, (ii) inputting the certain multi-cancer score values into the patient clustering model and instructing the patient clustering model to output certain patient cluster information on which cluster the certain patient belongs to among the first cluster to the k-th cluster by using the certain multi-cancer values and (iii) inputting the certain biomarker group-related value information into the certain patient cancer classification model, corresponding to the certain patient cluster information, among the first patient cancer classification model to the k-th patient cancer classification model and instructing certain patient cancer classification model to output certain cancer information resulting from predicting multi-cancer by using the certain biomarker group-related value information.

As one example, at the process of (I), the diagnosis model generation device has performed processes of (i) inputting each piece of the biomarker group-related value information from the training data into an initial classification model designed to classify multi-cancer by using the biomarker group-related value information, (ii) instructing the initial classification model to output first multi-cancer score values resulting from predicting multi-cancer by using each of the biomarker group-related value information, (iii) training the initial classification model by using a loss function acquired by referring to (iii-1) the first multi-cancer score values and (iii-2) each piece of the Ground Truth cancer information from the training data and thus (iv) generating the multi-cancer classification model.

As one example, at the process of (I), the diagnosis model generation device has performed processes of (i) inputting each of the biomarker group-related value information into the multi-cancer classification model and instructing the multi-cancer classification model to output second multi-cancer score values that predict multi-cancer on each of the biomarker group-related value information, (ii) inputting the second multi-cancer score values into an initial clustering model and instructing the initial clustering model to group the patients by using the second multi-cancer score values and (iii) instructing the initial clustering model to perform unsupervised learning to grouping the patients into k clusters by using an clustered distribution and thereby generates the patient clustering model.

As one example, at the process of (I), the diagnosis model generation device has performed process of (i) inputting second multi-cancer score values where multi-cancer is predicted for each of the biomarker group-related value information through the multi-cancer classification model into the patient clustering model and instructing the patient clustering model to group the patients into a first cluster to a k-th cluster by using second multi-cancer score values, (ii) instructing the patient clustering model to perform re-training by using each of the first data corresponding to first patients in the first cluster to k-th data corresponding to k-th patients in the k-th cluster, respectively, and thereby generates first patient cancer classification model to the k-th patient cancer classification model.

### BRIEF DESCRIPTION OF THE DRAWINGS

The above and other objects and features of the present invention will become apparent from the following description of preferred embodiments given in conjunction with the accompanying drawings, in which:

FIG. 1 is a drawing schematically illustrating a diagnosis model generation device capable of diagnosing multi-cancer by using biomarker group-related value information in accordance with one example embodiment of the present disclosure.

FIG. 2 is a drawing schematically illustrating a method for generating the diagnosis model capable of diagnosing multi-cancer by using the biomarker group-related value information in accordance with one example embodiment of the present disclosure.

FIG. 3 is a drawing schematically illustrating a multi-cancer diagnosis device capable of diagnosing multi-cancer by using the biomarker group-related value information in accordance with one example embodiment of the present disclosure.

FIG. 4 is a drawing schematically illustrating a method for diagnosing multi-cancer by using the biomarker group-related value information in accordance with one example embodiment of the present disclosure.

### DETAILED DESCRIPTION OF THE PREFERRED EMBODIMENTS

In the following detailed description, reference is made to the accompanying drawings that show, by way of illustration, specific embodiments in which the disclosure may be practiced. These embodiments are described in sufficient detail to enable those skilled in the art to practice the disclosure. It is to be understood that the various embodiments of the present disclosure, although different, are not necessarily mutually exclusive. For example, a particular feature, structure, or characteristic described herein in connection with one embodiment may be implemented within other embodiments without departing from the spirit and scope of the present disclosure. In addition, it is to be understood that the position or arrangement of individual elements within each disclosed embodiment may be modified without departing from the spirit and scope of the present disclosure. The following detailed description is, therefore, not to be taken in a limiting sense, and the scope of the present disclosure is defined only by the appended claims, appropriately interpreted, along with the full range of equivalents to which the claims are entitled. In the drawings, like numerals refer to the same or similar components throughout the several aspects.

To allow those skilled in the art to the present disclosure to be carried out easily, the example embodiments of the present disclosure by referring to attached diagrams will be explained in detail as shown below.

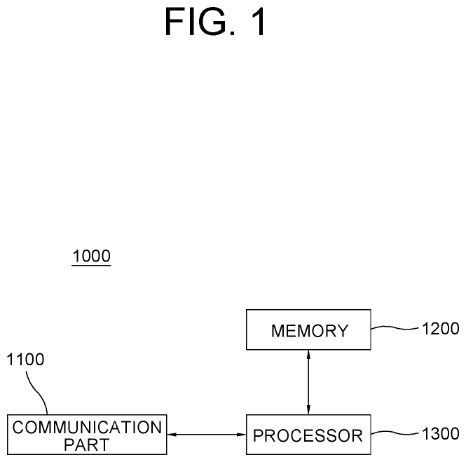

FIG. 1 is a drawing schematically illustrating a diagnosis model generation device capable of diagnosing multi-cancer by using biomarker group-related value information in accordance with one example embodiment of the present disclosure.

Referring to FIG. 1, the diagnosis model generation device 1000 in accordance with one example embodiment of the present disclosure may include (i) a communication part 1100 for acquiring n pieces of training data including the biomarker group-related value information and its corresponding Ground Truth cancer information for each of patients, (ii) a memory 1200 that stores instructions for generating a diagnosis model capable of diagnosing multi-cancer by using the training data acquired from the communication part 1100 and (iii) a processor 1300 configured to execute the instructions to perform processes of generating the diagnosis model by using the training data.

Herein, the communication part 1100 may acquire the training data from other devices storing the training data or from a user input.

Specifically, the diagnosis model generation device 1000 may achieve desired system performance by using combinations of a computing device, e.g., a computer processor, a memory, a storage, an input device, an output device, and other devices that may include components of conventional computing devices; an electronic communication device such as a router or a switch; an electronic information storage system such as a network-attached storage (NAS) device and a storage area network (SAN), and computer software, i.e., instructions that allow the computing device to function in a specific way.

The processor of such devices may include hardware configuration of MPU (Micro Processing Unit) or CPU (Central Processing Unit), cache memory, data bus, etc. Additionally, OS and software configuration of applications that achieve specific purposes may be further included.

However, the computing device does not exclude an integrated device including any combination of a processor, a memory, a medium, or any other computing components for implementing the present disclosure.

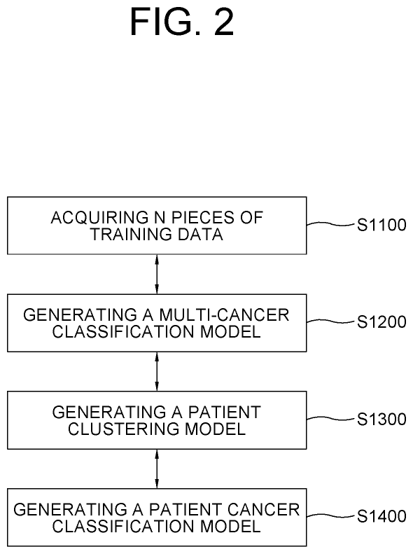

A method for generating the diagnosis model capable of diagnosing multi-cancer by using the biomarker group-related value information in accordance with one example embodiment of the present disclosure is described with FIG. 2.

First, the diagnosis model generation device 1000 may acquire n pieces of training data at a step of S1100.

As an example, the diagnosis model generation device 1000 may acquire the training data from other devices storing the training data or from a user input.

Herein, the training data may include the biomarker group-related value information and its corresponding Ground Truth cancer information for each of patients. Further, the Ground Truth cancer information for each of the patients may be actual cancer information and the number of cancer information is one or more.

Meanwhile, the biomarker group-related value information may be value information related to each of biomarkers. Herein, a biomarker may be an indicator value for diagnosing a cancer by using proteins, nucleic acids, and metabolites contained in a biological sample such as blood, urine, etc. derived from a patient's body. Also, the training data may include information, such as age, gender and medical history, etc., for each of the patients.

Next, the diagnosis model generation device 1000 may generate a multi-cancer classification model that classifies multi-cancer corresponding to the biomarker group-related value information by using the training data at a step of S1200.

That is, the diagnosis model generation device 1000 may (i) input each piece of the biomarker group-related value information from the training data into an initial classification model designed to classify multi-cancer by using the biomarker group-related value information, (ii) instruct the initial classification model to output first multi-cancer score values resulting from predicting multi-cancer by using each of the biomarker group-related value information, (iii) train the initial classification model by using a loss function acquired by referring to (iii-1) the first multi-cancer score values and (iii-2) each piece of the Ground Truth cancer information of the training data and thus (iv) generate the multi-cancer classification model.

Herein, the initial classification model may be any one of a decision tree model, a tree ensemble model, a random forest model, Bayesian network model, support vector machine model, neural network model or logistic regression model and the initial classification model may be (i) designed in advance and stored in a memory or (ii) stored in another computing device interacting with the diagnosis model generation device 1000.

A method of the diagnosis model generation device 1000 generating the multi-cancer classification model by training the initial classification model with the training data is described more specifically as below.

For the training data, (i) input variables which are the biomarker group-related value information for each of the patients and (ii) output variables which are score values corresponding to each of cancers resulting from predicting the biomarker group-related value information can be defined as follows.

| | |
|---|---|
| xᵢ ∈ R^{p} | i ∈ {1, ..., n} |
| yᵢ ∈ {1, ..., L} | i ∈ {1, ..., n} |

L may depict the number of cancers included in the multi-cancer to be classified.

In case the initial classification model is a logistic regression, that is, a multinomial linear logistic regression model, a function of the model may be depicted as follows.

$${\hat{f}}^{( 0 )} ( {x}_{i} ) = ( \begin{matrix}{{\hat{f}}^{( 0 )} ( {x}_{i} )}_{( 1 )} \\ … \\ {{\hat{f}}^{( 0 )} ( {x}_{i} )}_{( L )}\end{matrix} ) = {\hat{W}}^{( 0 )} {x}_{i} + {\hat{b}}^{( 0 )} i ∈ \{ 1 , … , n \}$$

Also, probabilities for the output variables may be depicted as follows.

$${\hat{P}}_{r}^{( 0 )} ( {y}_{i} = l ) = \frac{{{e}^{{\hat{f}}^{( 0 )}} ( {x}_{i} )}_{( l )}}{{{e}^{{\hat{f}}^{( 0 )}} ( {x}_{i} )}_{( l )} + … + {{e}^{{\hat{f}}^{( 0 )}} ( {x}_{i} )}_{( L )}} i ∈ \{ 1 , … , L \}$$

And, a loss function may be depicted as follows.

$$L ( {\hat{W}}^{( 0 )} , {\hat{b}}^{( 0 )} ) = - \frac{1}{n} {\sum }_{i = 1}^{n} {\sum }_{l = 1}^{L} I ( {y}_{i} = l ) log ( {\hat{P}}_{r}^{( 0 )} ( {y}_{i} = l ) )$$

Therefore, by finding Ŵ⁽⁰⁾∈ R^{L×p}, \hat{b}⁽⁰⁾∈R^{L} that minimize the loss function L(Ŵ^{(k)},\hat{b}^{(k)}) by using the n pieces of training data, the diagnosis model generation device 1000 may generate the multi-cancer classification model capable of classifying multi-cancer by referring to the biomarker group-related information. Herein, W may be weight parameters of the model and b may be bias parameters of the model.

Meanwhile, in case the initial classification model is a neural network model, a function of the model may be depicted as follows.

$${\hat{f}}^{( 0 )} ( {x}_{i} ) = ( \begin{matrix}{{\hat{f}}^{( 0 )} ( {x}_{i} )}_{( 1 )} \\ … \\ {{\hat{f}}^{( 0 )} ( {x}_{i} )}_{( L )}\end{matrix} ) = {\hat{W}}_{2}^{( 0 )} σ ( {\hat{W}}_{1}^{( 0 )} {x}_{i} + {\hat{b}}_{1}^{( 0 )} ) + \begin{matrix}{\hat{b}}_{2}^{( 0 )} & i ∈ \{ 1 , … , n \}\end{matrix}$$

Herein, σ(.) may be an activation function in a hidden layer at the neural network model. Further, the activation function may be any one of sigmoid function, hyperbolic tangent function, and rectified linear unit function (ReLU), but it is not limited thereto. Also, there is one hidden layer as an example, but the scope of the present disclosure is not limited thereto, the number of the hidden layer is one or more.

Also, probabilities for the output variables may be depicted as follows.

$${\hat{P}}_{r}^{( 0 )} ( {y}_{i} = l ) = \begin{matrix}\frac{{e}^{{{\hat{f}}^{( 0 )} ( {x}_{i} )}_{( l )}}}{{e}^{{{\hat{f}}^{( 0 )} ( {x}_{i} )}_{( 1 )}} + … + {e}^{{{\hat{f}}^{( 0 )} ( {x}_{i} )}_{( L )}}} & l ∈ \{ 1 , … , L \}\end{matrix}$$

And, a loss function can be depicted as follows.

$$L ( {\hat{W}}_{1}^{( 0 )} , {\hat{b}}_{1}^{( 0 )} , {\hat{W}}_{2}^{( 0 )} , {\hat{b}}_{2}^{( 0 )} ) = - \frac{1}{n} {\sum }_{i = 1}^{n} {\sum }_{l = 1}^{L} I ( {y}_{i} = l ) log ( {\hat{P}}_{r}^{( 0 )} ( {y}_{i} = l ) )$$

Therefore, the diagnosis model generation device 1000 may generate the multi-cancer classification model with updated bias parameters (b₁,b₂) and updated weight parameters (W₁,W₂) to minimize the loss function L(Ŵ₁⁽⁰⁾,\hat{b}₁⁽⁰⁾,Ŵ₂⁽⁰⁾,\hat{b}₂⁽⁰⁾) at the initial classification model.

Next, the diagnosis model generation device 1000 may generate a patient clustering model capable of classifying the patients into any of k clusters at a step of S1300 by referring to multi-cancer score values outputted from the multi-cancer classification model. Herein, k is an integer of 1 or more. Further, the multi-cancer score values represent results of classifying multi-cancer by using the biomarker group-related value information.

That is, the diagnosis model generation device 1000 (i) inputs each of the biomarker group-related value information on each of the patients into the multi-cancer classification model and instructs the multi-cancer classification model to output second multi-cancer score values resulting from predicting multi-cancer by using each of the biomarker group-related value information, (ii) inputs the second multi-cancer score values into an initial clustering model and instructs the initial clustering model to group the patients by using the second multi-cancer score values and (iii) instructs the initial clustering model to perform unsupervised learning for grouping the patients into k clusters by using a clustered distribution and thus generates the patient clustering model.

Herein, the initial classification model is any one of a K-Means Clustering model, a Mean-Shift Clustering model, a Density-Based Spatial Clustering of Applications with Noise (DBSCAN) model, an Expectation-Maximization (EM) model, Clustering using Gaussian Mixture Models (GMM), or an Agglomerative Hierarchical Clustering model. Further, the initial clustering model may be (i) designed in advance and stored in a memory or (ii) stored in another computing device interacting with the diagnosis model generation device 1000.

As an example, in case an initial clustering model is K-means clustering model capable of selecting nearest data from a center, i.e., a centroid, selected randomly in a certain random point, an unsupervised learning can be performed by repeating following processes of (i) moving the centroid to an average point between the centroid and the selected nearest data, (ii) selecting its corresponding nearest data from the average point. In case the number of repetition is larger than a predefined number of the repetition or in case a distance moved from its corresponding centroid to its corresponding new average point is smaller than a predefined convergence criterion, the repetition may be completed.

As another example, in case an initial clustering model is an Agglomerative Hierarchical Clustering model, unsupervised learning can be performed by performing processes of, as an example, (i) initializing each of data points as each of single clusters, (ii) calculating distance metrics such as average linkage defined as average distance between data points in a first cluster and data points in a second cluster (iii) combining two clusters which have the smallest average linkage repetitively until reaching at the root of dendrogram and (iv) selecting a point of time to stop clustering, that is, the point of time to stop generating the dendrogram, to thereby determine the number of clusters.

Clusters grouped by this kind of clustering model can be depicted as follows. That is, n pieces of training data {x₁, . . . , xₙ} can be grouped into k clusters S={S₁, . . . , S_{K}} by using score values \hat{f}⁽⁰⁾(xᵢ) of the multi-cancer diagnosis model

$$S = {arg min}_{S} {\sum }_{k = 1}^{K} {\sum }_{{x}_{i} ∈ {S}_{k}} {‖ {\hat{f}}^{( 0 )} ( {x}_{i} ) - {μ}_{k} ‖}^{2}$$

Herein, μₖ may be a centroid of the cluster Sₖ.

And, clusters C(zᵢ), corresponding to patients inputted by the trained clustering model, may be assigned as follows.

$$C ( {z}_{i} ) = {arg min}_{k ∈ \{ 1 , … , K \}} ‖ {\hat{f}}^{( 0 )} ( {z}_{i} ) - {μ}_{k} ‖$$

Meanwhile, clusters grouped by the clustering model can be represented as follows. A table below represents a result of grouping five cancers into six clusters as an example.

| | Cancer 1 | Cancer 2 | Cancer 3 | Cancer 4 | Cancer 5 |
|---|---|---|---|---|---|
| Cluster 1 | 1.0074452 | 0.16029899 | 1.2470591 | −2.0800545 | −0.04665457 |
| Cluster 2 | −0.02240956 | −0.24331826 | −1.1085218 | 1.7155764 | −0.51330817 |
| Cluster 3 | 0.04779798 | 0.9791689 | −0.08155832 | −0.37238878 | 0.44095463 |
| Cluster 4 | 1.2745931 | −0.17150354 | 0.50528294 | −0.2911493 | 0.9430351 |
| Cluster 5 | −0.40799809 | 2.515143 | −0.34812155 | −0.8898516 | −1.4215368 |
| Cluster 6 | 1.1775391 | −2.0019574 | 1.3806248 | −1.2684903 | −1.9313437 |

As represented in the table above, the cluster 1 represents a patient group with a high probability of the cancer 1 and the cancer 3 and a low probability of the cancer 4, the cluster 2 represents a patient group with a high probability of the cancer 4 and a low probability of the cancer 3, the cluster 3 represents a patient group with a high probability of the cancer 2 and a high probability of the cancer 5, the cluster 4 represents a patient group with a high probability of the cancer 1 and a high probability of the cancer 5, the cluster 5 represents a patient group with a very high probability of the cancer 2 and the cluster 6 represents a patient group with a high probability of the cancer 1 and a high probability of the cancer 3.

Next, the diagnosis model generation device 1000 may (i) re-train the multi-cancer classification model by using each of partial training data corresponding to each of the k clusters grouped by the patient clustering model, wherein the partial training data is a part of the training data, thereby generating a first patient cancer classification model to a k-th patient cancer classification model and thus (ii) generate a diagnosis model including the multi-cancer classification model, the patient clustering model and the first patient cancer classification model to the k-th patient cancer classification model at a step of S1400.

That is, the diagnosis model generation device 1000 may (i) input second multi-cancer score values resulting from predicting multi-cancer by using each of the biomarker group-related value information through the multi-cancer classification model into the patient clustering model and instructs the patient clustering model to group the patients into a first cluster to a k-th cluster by using the second multi-cancer score values and (ii) instruct the patient clustering model to perform re-training by using each of first data corresponding to first patients in the first cluster to k-th data corresponding to k-th patients in the k-th cluster, respectively, and thereby generate the first patient cancer classification model to the k-th patient cancer classification model.

A method of the diagnosis model generation device 1000 generating the first patient cancer classification model to the k-th patient cancer classification model on the basis of the multi-cancer classification model by using the training data for each of the clusters may be described specifically as follows.

Let the number of the training data on the patients belonging to a cluster k∈{1, . . . , K} grouped by the patients clustering model is nₖ. Then, input variables and output variables may be defined as follows.

| | |
|---|---|
| xₖᵢ ∈ R^{p} | i ∈ {1, ..., nₖ} |
| yₖᵢ ∈ {1, ..., L} | i ∈ {1, ..., nₖ} |

Herein, L may be the number of multi-cancer to be classified.

Herein, in case the multi-cancer classification model is a logistic regression model, that is, multinomial linear logistic regression model, a function of the model may be depicted as follows.

$${\hat{f}}^{( k )} ( {x}_{ki} ) = ( \begin{matrix}{{\hat{f}}^{( k )} ( {x}_{ki} )}_{( 1 )} \\ … \\ {{\hat{f}}^{( k )} ( {x}_{ki} )}_{( L )}\end{matrix} ) = {\hat{W}}^{( k )} {x}_{ki} + \begin{matrix}{\hat{b}}^{( k )} & i ∈ \{ 1 , … , {n}_{k} \}\end{matrix}$$

Also, probabilities for the output variables may be depicted as follows.

$${\hat{P}}_{r}^{( k )} ( {y}_{ki} = l ) = \begin{matrix}\frac{{e}^{{{\hat{f}}^{( k )} ( {x}_{ki} )}_{( l )}}}{{e}^{{{\hat{f}}^{( k )} ( {x}_{ki} )}_{( 1 )}} + … + {e}^{{{\hat{f}}^{( k )} ( {x}_{ki} )}_{( L )}}} & i ∈ \{ 1 , … , L \}\end{matrix}$$

And, a loss function may be depicted as follows.

$${L}_{1} ( {\hat{W}}^{( k )} , {\hat{b}}^{( k )} ) = - \frac{1}{{n}_{k}} {\sum }_{i = 1}^{{n}_{k}} {\sum }_{l = 1}^{L} I ( {y}_{ki} = l ) log ( {\hat{P}}_{r}^{( k )} ( {y}_{ki} = l ) ) + {λ}_{1} ( {‖ {\hat{W}}^{( k )} - {\hat{W}}^{( 0 )} ‖}_{F}^{2} + {‖ {\hat{b}}^{( k )} - {\hat{b}}^{( 0 )} ‖}_{2}^{2} )$$

Therefore, by finding Ŵ^{(k)}∈R^{L×p}, \hat{b}^{(k)}∈R^{L} that minimize the loss function L(Ŵ^{(k)},\hat{b}^{(k)}) for a tuning parameter λ₁ by using nₖ pieces of the training data, the diagnosis model generation device 1000 may generate a first patient cancer classification model to a k-th patient cancer classification model as a result of re-training the multi-cancer classification model with the training data for each of the clusters. Herein, by setting the tuning parameter λ₁ as small value, 0.1 as an example, a balance between a bias and a variance can be optimized.

Meanwhile, in case the multi-cancer classification model is a neural network model, a function of model may be depicted as follows.

$${\hat{f}}^{( k )} ( {x}_{ki} ) = ( \begin{matrix}{{\hat{f}}^{( k )} ( {x}_{ki} )}_{( 1 )} \\ … \\ {{\hat{f}}^{( k )} ( {x}_{ki} )}_{( L )}\end{matrix} ) = {\hat{W}}_{2}^{( k )} σ ( {\hat{W}}_{1}^{( k )} {x}_{ki} + {\hat{b}}_{1}^{( k )} ) + \begin{matrix}{\hat{b}}_{2}^{( k )} & i ∈ \{ 1 , … , {n}_{k} \}\end{matrix}$$

Herein, σ(.) may be an activation function in a hidden layer at the neural network model, any one of sigmoid function, hyperbolic tangent function, and rectified linear unit function (ReLU) can be used as the activate function but it is not limited thereto. Also, there is one hidden layer as an example above, but it is not limited thereto, the number of the hidden layer may be one or more.

Also, probabilities for the output variables may be depicted as follows.

$${\hat{P}}_{r}^{( k )} ( {y}_{ki} = l ) = \begin{matrix}\frac{{e}^{{{\hat{f}}^{( k )} ( {x}_{ki} )}_{( l )}}}{{e}^{{{\hat{f}}^{( k )} ( {x}_{ki} )}_{( 1 )}} + … + {e}^{{{\hat{f}}^{( k )} ( {x}_{ki} )}_{( L )}}} & i ∈ \{ 1 , … , L \}\end{matrix}$$

And, a loss function can be depicted as follows.

$${L}_{2} ( {\hat{W}}_{1}^{( k )} , {\hat{b}}_{1}^{( k )} , {\hat{W}}_{2}^{( k )} , {\hat{b}}_{2}^{( k )} ) = - \frac{1}{n} {\sum }_{i = 1}^{{n}_{k}} {\sum }_{l = 1}^{L} I ( {y}_{ki} = l ) log ( {\hat{P}}_{r}^{( k )} ( {y}_{ki} = l ) ) + {λ}_{2} ( {‖ {\hat{W}}_{1}^{( k )} - {\hat{W}}_{1}^{( 0 )} ‖}_{F}^{2} + {‖ {\hat{W}}_{2}^{( k )} - {\hat{W}}_{2}^{( 0 )} ‖}_{F}^{2} + {‖ {\hat{b}}_{1}^{( k )} - {\hat{b}}_{1}^{( 0 )} ‖}_{2}^{2} + {‖ {\hat{b}}_{2}^{( k )} - {\hat{b}}_{2}^{( 0 )} ‖}_{2}^{2} )$$

Therefore, the diagnosis model generation device 1000 may generate a first patient cancer classification model to a k-th patient cancer classification model with updated bias parameters (b₁,b₂) and updated weight parameters (W₁,W₂) by updating bias parameters and weight parameters of the multi-cancer classification model. Herein, the bias parameters and the weight parameters of the multi-cancer classification model are updated by using the training data for each of the clusters to minimize loss function L(W₁^{(k)},\hat{b}₁^{(k)},Ŵ₂^{(k)},\hat{b}₂^{(k)}) with a tuning parameter λ₂. Herein, by setting the tuning parameter λ₂ as small value, 0.1 as an example, the balance between the bias and the variance can be optimized.

On the condition that the diagnosis model for diagnosing multi-cancer by using biomarker group-related value information has been generated, a diagnosing method and a diagnosing device using the biomarker group-related value information are described in detail as below.

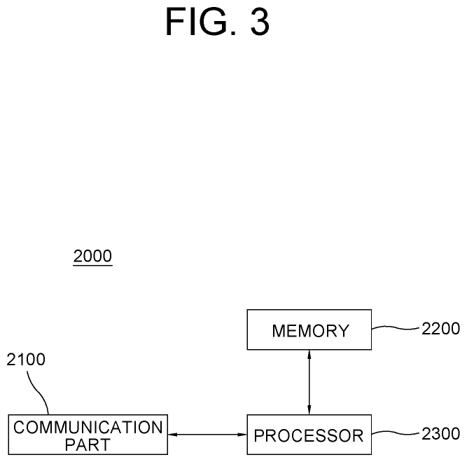

FIG. 3 is a drawing schematically illustrating a multi-cancer diagnosis device capable of diagnosing multi-cancer by using the biomarker group-related value information in accordance with one example embodiment of the present disclosure.

Referring to FIG. 3, a multi-cancer diagnosis device 2000 in accordance with one example embodiment of the present disclosure may include (i) a communication part 2100 for acquiring the biomarker group-related value information corresponding to a certain patient, (ii) a memory 2200 for storing instructions for diagnosing multi-cancer by using the biomarker group-related value information acquired from the communication part 2100 and (iii) a processor 2300 for performing operations of diagnosing multi-cancer by using the biomarker group-related value information of the certain patient according to the instructions stored in the memory 2200.

Herein, the communication part 2100 may acquire (i) the biomarker group-related value information of the certain patient from another device that has generated the biomarker group-related value information of the certain patient and (ii) the biomarker group-related value information of the certain patient as a user input.

Specifically, the multi-cancer diagnosis device 2000 may achieve desired system performance by using combinations of a computing device, e.g., a computer processor, a memory, a storage, an input device, an output device, and other devices that may include components of conventional computing devices; an electronic communication device such as a router or a switch; an electronic information storage system such as a network-attached storage (NAS) device and a storage area network (SAN), and computer software, i.e., instructions that allow the computing device to function in a specific way.

The processor of such devices may include hardware configuration of MPU (Micro Processing Unit) or CPU (Central Processing Unit), cache memory, data bus, etc. Additionally, OS and software configuration of applications that achieve specific purposes may be further included.

However, the computing device does not exclude an integrated device including any combination of a processor, a memory, a medium, or any other computing components for implementing the present disclosure.

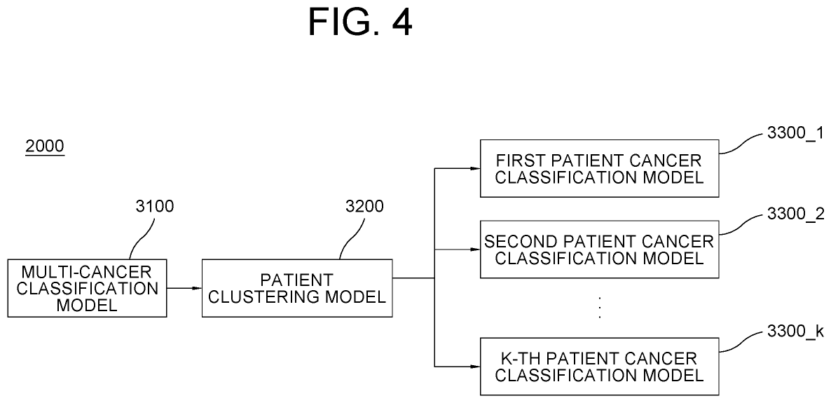

A method of the multi-cancer diagnosis device diagnosing multi-cancer by using the biomarker group-related value information in accordance with one example embodiment of the present disclosure is described by referring to FIG. 4.

First, according to the detailed description above related to FIG. 2, on the condition that the multi-cancer classification model 3100, the patient clustering model 3200 and the first patient cancer classification model 3300_1 to the k-th patient cancer classification model 3300_k have been generated, the multi-cancer diagnosis device 2000 may acquire certain biomarker group-related value information of the certain patient to diagnose multi-cancer.

As an example, the multi-cancer diagnosis device 2000 may acquire the certain biomarker group-related value information of the certain patient by interacting with another device that has generated the biomarker group-related value information of the certain patient or from the user input.

Next, the multi-cancer diagnosis device 2000 may input the certain biomarker group-related value information of the certain patient into the multi-cancer classification model 3100.

Then, the multi-cancer classification model 3100 may output certain multi-cancer score values resulting from predicting multi-cancer by using the certain biomarker group-related value information of the certain patient.

Herein, in case the multi-cancer classification model 3100 is multinomial logistic regression model, the multi-cancer classification model 3100 may output the certain multi-cancer score values that are resulted from predicting multi-cancer by using the certain biomarker group-related value information z as follows.

$${\hat{f}}^{( 0 )} ( z ) = ( \begin{matrix}{{\hat{f}}^{( 0 )} ( z )}_{( 1 )} \\ … \\ {{\hat{f}}^{( 0 )} ( z )}_{( L )}\end{matrix} ) = {\hat{W}}^{( 0 )} z + {\hat{b}}^{( 0 )}$$

And, in case the multi-cancer classification model 3100 is neural network model, the multi-cancer classification model 3100 may output the certain multi-cancer score values that are resulted from predicting multi-cancer by using the certain biomarker group-related value information z as follows.

$${\hat{f}}^{( 0 )} ( z ) = ( \begin{matrix}{{\hat{f}}^{( 0 )} ( z )}_{( 1 )} \\ … \\ {{\hat{f}}^{( 0 )} ( z )}_{( L )}\end{matrix} ) = {\hat{W}}_{2}^{( 0 )} σ ( {\hat{W}}_{1}^{( 0 )} z + {\hat{b}}_{1}^{( 0 )} ) + {\hat{b}}_{2}^{( 0 )}$$

Next, the multi-cancer diagnosis device 2000 may input the certain multi-cancer score values, outputted from the multi-cancer classification model 3100, into the patient clustering model 3200.

Then, the patient clustering model 3200 may output information on which cluster the certain patients belongs to among the first cluster to the k-th cluster by using the certain multi-cancer score values.

Herein, a certain cluster C(z) to which the certain patients belongs in accordance with a certain multi-cancer score values \hat{f}⁽⁰⁾(z) can be defined as follows.

$$C ( z ) = {arg min}_{k ∈ \{ 1 , … , K \}} ‖ {\hat{f}}^{( 0 )} ( z ) - {μ}_{k} ‖$$

Next, the multi-cancer diagnosis device 2000 may input the certain biomarker group-related value information of the certain patient z into a certain patient cancer classification model, corresponding to the certain cluster C(z), among the first patient cancer classification model 3300_1 to the k-th patient cancer classification model 3300_k.

Then, the certain patient cancer classification model may analyze the certain biomarker group-related value information of the certain patient z and output certain cancer information on the certain patient.

Herein, the certain cancer information, outputted by the certain patient cancer classification model, can be depicted as follows.

$$y ( z ) = {arg min}_{k ∈ \{ 1 , … , L \}} {{\hat{f}}^{( C ( z ) )} ( z )}_{( l )}$$

Therefore, in accordance with the present disclosure, the first patient cancer classification model to the k-th patient cancer classification model are generated on the basis of the multi-cancer classification model (having been trained by using total training data) by re-training the multi-cancer classification model for each of the clusters, and thus balance between the bias and the variance can be optimized and multi-cancer can be diagnosed more accurately.

The present disclosure has an effect of providing a diagnosis model that minimizes the bias and the variance by using biomarker group-related value information for each of patients.

The present disclosure has another effect of allowing a type of cancer to be predicted accurately by using the biomarker group-related value information for multi-cancer diagnosis.

The present disclosure has still another effect of allowing a type of cancer to be predicted accurately through a statistical discriminant analysis using biomarker group-related value information for the multi-cancer diagnosis.

The present disclosure has still yet another effect of increasing a credibility of cancer diagnosis through the statistical discriminant analysis using the biomarker group-related value information for the multi-cancer diagnosis.

The embodiments of the present invention as explained above can be implemented in a form of executable program command through a variety of computer means recordable to computer readable media. The computer readable media may include solely or in combination, program commands, data files, and data structures. The program commands recorded to the media may be components specially designed for the present invention or may be usable to a skilled human in a field of computer software. Computer readable media include magnetic media such as hard disk, floppy disk, and magnetic tape, optical media such as CD-ROM and DVD, magneto-optical media such as floptical disk and hardware devices such as ROM, RAM, and flash memory specially designed to store and carry out program commands. Program commands include not only a machine language code made by a complier but also a high level code that can be used by an interpreter etc., which is executed by a computer. The aforementioned hardware device can work as more than a software module to perform the action of the present invention and they can do the same in the opposite case.

As seen above, the present invention has been explained by specific matters such as detailed components, limited embodiments, and drawings. They have been provided only to help more general understanding of the present invention. It, however, will be understood by those skilled in the art that various changes and modification may be made from the description without departing from the spirit and scope of the invention as defined in the following claims.

Accordingly, the thought of the present invention must not be confined to the explained embodiments, and the following patent claims as well as everything including variations equal or equivalent to the patent claims pertain to the category of the thought of the present invention.

1. A method for generating diagnosis model capable of diagnosing multi-cancer using biomarker group-related value information comprising steps of:
(a) a diagnosis model generation device, in response to acquiring n pieces of training data including the biomarker group-related value information and its corresponding Ground Truth cancer information for each of patients, generating a multi-cancer classification model that classifies multi-cancer corresponding to the biomarker group-related value information by using the n pieces of the training data, wherein n is an integer of 1 or more, and wherein the multi-cancer classification model is a single classification model for classifying each of the multi-cancer of each of the patients;
(b) the diagnosis model generation device generating a patient clustering model which classifies the patients into any of k clusters, wherein the k is an integer of 1 or more, by referring to multi-cancer score values outputted from the multi-cancer classification model, wherein the multi-cancer score values represent results of classifying multi-cancer by using the biomarker group-related value information; and
(c) the diagnosis model generation device (i) re-training the multi-cancer classification model such that weight parameters and bias parameters of the multi-cancer classification model are fine-tuned by a tuning parameter by using each of partial training data corresponding to each of the k clusters grouped by the patient clustering model, wherein the partial training data is a part of the n pieces of the training data, thereby generating a first patient cancer classification model to a k-th patient cancer classification model and thus (ii) generating a diagnosis model including the multi-cancer classification model, the patient clustering model and the first patient cancer classification model to the k-th patient cancer classification model.

2. The method of claim 1, wherein, at the step of (a), the diagnosis model generation device (i) inputs each piece of the biomarker group-related value information from the training data into an initial classification model designed to classify multi-cancer by using the biomarker group-related value information, (ii) instructs the initial classification model to output first multi-cancer score values resulting from predicting multi-cancer by using each of the biomarker group-related value information, (iii) trains the initial classification model by using a loss function acquired by referring to (iii-1) the first multi-cancer score values and (iii-2) each piece of the Ground Truth cancer information of the training data and thus (iv) generates the multi-cancer classification model.

3. The method of claim 2, wherein the initial classification model is any one of a decision tree model, a tree ensemble model, a random forest model, Bayesian network model, support vector machine model, neural network model or logistic regression model.

4. The method of claim 1, wherein, at the step of (b), the diagnosis model generation device (i) inputs each of the biomarker group-related value information on each of the patients into the multi-cancer classification model and instructs the multi-cancer classification model to output second multi-cancer score values resulting from predicting multi-cancer by using each of the biomarker group-related value information, (ii) inputs the second multi-cancer score values into an initial clustering model and instructs the initial clustering model to group the patients by using the second multi-cancer score values and (iii) instructs the initial clustering model to perform unsupervised learning for grouping the patients into k clusters by using a clustered distribution and thus generates the patient clustering model.

5. The method of claim 4, wherein the initial classification model is any one of a K-Means Clustering model, a Mean-Shift Clustering model, a Density-Based Spatial Clustering of Applications with Noise (DBSCAN) model, an Expectation-Maximization (EM) model, Clustering using Gaussian Mixture Models (GMM), or an Agglomerative Hierarchical Clustering model.

6. The method of claim 1, wherein, at the step of (c), the diagnosis model generation device (i) inputs second multi-cancer score values resulting from predicting multi-cancer by using each of the biomarker group-related value information through the multi-cancer classification model into the patient clustering model and instructs the patient clustering model to group the patients into a first cluster to a k-th cluster by using the second multi-cancer score values and (ii) instructs the patient clustering model to perform re-training by using each of first data corresponding to first patients in the first cluster to k-th data corresponding to k-th patients in the k-th cluster, respectively, and thereby generates the first patient cancer classification model to the k-th patient cancer classification model.

7. A method for diagnosing multi-cancer using biomarker group-related value information comprising steps of:
(a) on condition that the diagnosis model generation device, (i) in response to acquiring n pieces of training data including the biomarker group-related value information and its corresponding Ground Truth cancer information for each of patients, has generated a multi-cancer classification model that classifies multi-cancer corresponding to the biomarker group-related value information by using the n pieces of the training data, wherein n is an integer of 1 or more, and wherein the multi-cancer classification model is a single classification model for classifying each of the multi-cancer of each of the patients (ii) has generated a patient clustering model which classifies the patients into any of k clusters, wherein the k is an integer of 1 or more, by referring to multi-cancer score values outputted from the multi-cancer classification model, wherein the multi-cancer score values represent results of classifying multi-cancer by using the biomarker group-related value information and (iii) (iii-1) has re-trained the multi-cancer classification model by using each of partial training data corresponding to each of the k clusters grouped by the patient clustering model, wherein the partial training data is a part of the n pieces of the training data, (iii-2) has generated a first patient cancer classification model to a k-th patient cancer classification model and thus (iii-3) has generated a diagnosis model including the multi-cancer classification model, the patient clustering model and the first patient cancer classification model to the k-th patient cancer classification model, a multi-cancer diagnosis device acquiring certain biomarker group-related value information on a certain patient; and
(b) the multi-cancer diagnosis device (i) inputting the certain biomarker group-related value information into the multi-cancer classification model and instructing the multi-cancer classification model to output certain multi-cancer score values resulting from predicting multi-cancer by using the certain biomarker group-related value information, (ii) inputting the certain multi-cancer score values into the patient clustering model and instructing the patient clustering model to output certain patient cluster information on which cluster the certain patient belongs to among the first cluster to the k-th cluster by using the certain multi-cancer values and (iii) inputting the certain biomarker group-related value information into the certain patient cancer classification model, corresponding to the certain patient cluster information, among the first patient cancer classification model to the k-th patient cancer classification model and instructing certain patient cancer classification model to output certain cancer information resulting from predicting multi-cancer by using the certain biomarker group-related value information.

8. The method of claim 7, wherein, at the step of (a), the diagnosis model generation device has performed processes of (i) inputting each piece of the biomarker group-related value information from the training data into an initial classification model designed to classify multi-cancer by using the biomarker group-related value information, (ii) instructing the initial classification model to output first multi-cancer score values resulting from predicting multi-cancer by using each of the biomarker group-related value information, (iii) training the initial classification model by using a loss function acquired by referring to (iii-1) the first multi-cancer score values and (iii-2) each piece of the Ground Truth cancer information from the training data and thus (iv) generating the multi-cancer classification model.

9. The method of claim 7, wherein, at the step of (a), the diagnosis model generation device has performed processes of (i) inputting each of the biomarker group-related value information into the multi-cancer classification model and instructing the multi-cancer classification model to output second multi-cancer score values that predict multi-cancer on each of the biomarker group-related value information, (ii) inputting the second multi-cancer score values into an initial clustering model and instructing the initial clustering model to group the patients by using the second multi-cancer score values and (iii) instructing the initial clustering model to perform unsupervised learning to grouping the patients into k clusters by using an clustered distribution and thereby generates the patient clustering model.

10. The method of claim 7, wherein, at the step of (a), the diagnosis model generation device has performed process of (i) inputting second multi-cancer score values where multi-cancer is predicted for each of the biomarker group-related value information through the multi-cancer classification model into the patient clustering model and instructing the patient clustering model to group the patients into a first cluster to a k-th cluster by using second multi-cancer score values, (ii) instructing the patient clustering model to perform re-training by using each of the first data corresponding to first patients in the first cluster to k-th data corresponding to k-th patients in the k-th cluster, respectively, and thereby generates first patient cancer classification model to the k-th patient cancer classification model.

11. A diagnosis model generation device capable of diagnosing multi-cancer using biomarker group-related value information comprising:
one or more memories that stores instructions; and
one or more processors configured to execute the instructions to perform processes of (I) in response to acquiring n pieces of training data including the biomarker group-related value information and its corresponding Ground Truth cancer information for each of patients, generating a multi-cancer classification model that classifies multi-cancer corresponding to the biomarker group-related value information by using the n pieces of the training data, wherein n is an integer of 1 or more, and wherein the multi-cancer classification model is a single classification model for classifying each of the multi-cancer of each of the patients; (II) generating a patient clustering model which classifies the patients into any of k clusters, wherein the k is an integer of 1 or more, by referring to multi-cancer score values outputted from the multi-cancer classification model, wherein the multi-cancer score values represent results of classifying multi-cancer by using the biomarker group-related value information; and (III) (i) re-training the multi-cancer classification model such that weight parameters and bias parameters of the multi-cancer classification model are fine-tuned by a tuning parameter by using each of partial training data corresponding to each of the k clusters grouped by the patient clustering model, wherein the partial training data is a part of the n pieces of the training data, thereby generating a first patient cancer classification model to a k-th patient cancer classification model and thus (ii) generating a diagnosis model including the multi-cancer classification model, the patient clustering model and the first patient cancer classification model to the k-th patient cancer classification model.

12. The diagnosis model generation device of claim 11, wherein, at the process of (I), the processor (i) inputs each piece of the biomarker group-related value information from the training data into an initial classification model designed to classify multi-cancer by using the biomarker group-related value information, (ii) instructs the initial classification model to output first multi-cancer score values resulting from predicting multi-cancer by using each of the biomarker group-related value information, (iii) trains the initial classification model by using a loss function acquired by referring to (iii-1) the first multi-cancer score values and (iii-2) each piece of the Ground Truth cancer information of the training data and thus (iv) generates the multi-cancer classification model.

13. The diagnosis model generation device of claim 12, wherein the initial classification model is any one of a decision tree model, a tree ensemble model, a random forest model, Bayesian network model, support vector machine model, neural network model or logistic regression model.

14. The diagnosis model generation device of claim 11, wherein, at the process of (II), the processor (i) inputs each of the biomarker group-related value information on each of the patients into the multi-cancer classification model and instructs the multi-cancer classification model to output second multi-cancer score values resulting from predicting multi-cancer by using each of the biomarker group-related value information, (ii) inputs the second multi-cancer score values into an initial clustering model and instructs the initial clustering model to group the patients by using the second multi-cancer score values and (iii) instructs the initial clustering model to perform unsupervised learning for grouping the patients into k clusters by using a clustered distribution and thus generates the patient clustering model.

15. The diagnosis model generation device of claim 14, wherein the initial classification model is any one of a K-Means Clustering model, a Mean-Shift Clustering model, a Density-Based Spatial Clustering of Applications with Noise (DBSCAN) model, an Expectation-Maximization (EM) model, Clustering using Gaussian Mixture Models (GMM), or an Agglomerative Hierarchical Clustering model.

16. The diagnosis model generation device of claim 11, wherein, at the process of (III), the processor (i) inputs second multi-cancer score values resulting from predicting multi-cancer by using each of the biomarker group-related value information through the multi-cancer classification model into the patient clustering model and instructs the patient clustering model to group the patients into a first cluster to a k-th cluster by using the second multi-cancer score values and (ii) instructs the patient clustering model to perform re-training by using each of first data corresponding to first patients in the first cluster to k-th data corresponding to k-th patients in the k-th cluster, respectively, and thereby generates the first patient cancer classification model to the k-th patient cancer classification model.

17. A multi-cancer diagnosis device for diagnosing multi-cancer using biomarker group-related value information comprising:
one or more memories that stores instructions; and
one or more processors configured to execute the instructions to perform processes of (I) on condition that the diagnosis model generation device, (i) in response to acquiring n pieces of training data including the biomarker group-related value information and its corresponding Ground Truth cancer information for each of patients, has generated a multi-cancer classification model that classifies multi-cancer corresponding to the biomarker group-related value information by using the n pieces of the training data, wherein n is an integer of 1 or more, and wherein the multi-cancer classification model is a single classification model for classifying each of the multi-cancer of each of the patients (ii) has generated a patient clustering model which classifies the patients into any of k clusters, wherein the k is an integer of 1 or more, by referring to multi-cancer score values outputted from the multi-cancer classification model, wherein the multi-cancer score values represent results of classifying multi-cancer by using the biomarker group-related value information and (iii) (iii-1) has re-trained the multi-cancer classification model such that weight parameters and bias parameters of the multi-cancer classification model are fine-tuned by a tuning parameter by using each of partial training data corresponding to each of the k clusters grouped by the patient clustering model, wherein the partial training data is a part of the n pieces of the training data, (iii-2) has generated a first patient cancer classification model to a k-th patient cancer classification model and thus (iii-3) has generated a diagnosis model including the multi-cancer classification model, the patient clustering model and the first patient cancer classification model to the k-th patient cancer classification model, acquiring certain biomarker group-related value information on a certain patient; and (II) (i) inputting the certain biomarker group-related value information into the multi-cancer classification model and instructing the multi-cancer classification model to output certain multi-cancer score values resulting from predicting multi-cancer by using the certain biomarker group-related value information, (ii) inputting the certain multi-cancer score values into the patient clustering model and instructing the patient clustering model to output certain patient cluster information on which cluster the certain patient belongs to among the first cluster to the k-th cluster by using the certain multi-cancer values and (iii) inputting the certain biomarker group-related value information into the certain patient cancer classification model, corresponding to the certain patient cluster information, among the first patient cancer classification model to the k-th patient cancer classification model and instructing certain patient cancer classification model to output certain cancer information resulting from predicting multi-cancer by using the certain biomarker group-related value information.

18. The multi-cancer diagnosis device of claim 17, wherein, at the process of (I), the diagnosis model generation device has performed processes of (i) inputting each piece of the biomarker group-related value information from the training data into an initial classification model designed to classify multi-cancer by using the biomarker group-related value information, (ii) instructing the initial classification model to output first multi-cancer score values resulting from predicting multi-cancer by using each of the biomarker group-related value information, (iii) training the initial classification model by using a loss function acquired by referring to (iii-1) the first multi-cancer score values and (iii-2) each piece of the Ground Truth cancer information from the training data and thus (iv) generating the multi-cancer classification model.

19. The multi-cancer diagnosis device of claim 17, wherein, at the process of (I), the diagnosis model generation device has performed processes of (i) inputting each of the biomarker group-related value information into the multi-cancer classification model and instructing the multi-cancer classification model to output second multi-cancer score values that predict multi-cancer on each of the biomarker group-related value information, (ii) inputting the second multi-cancer score values into an initial clustering model and instructing the initial clustering model to group the patients by using the second multi-cancer score values and (iii) instructing the initial clustering model to perform unsupervised learning to grouping the patients into k clusters by using an clustered distribution and thereby generates the patient clustering model.

20. The multi-cancer diagnosis device of claim 17, wherein, at the process of (I), the diagnosis model generation device has performed process of (i) inputting second multi-cancer score values where multi-cancer is predicted for each of the biomarker group-related value information through the multi-cancer classification model into the patient clustering model and instructing the patient clustering model to group the patients into a first cluster to a k-th cluster by using second multi-cancer score values, (ii) instructing the patient clustering model to perform re-training by using each of the first data corresponding to first patients in the first cluster to k-th data corresponding to k-th patients in the k-th cluster, respectively, and thereby generates first patient cancer classification model to the k-th patient cancer classification model.
